(19) United States
(12) Patent Application Publication
(10) Pub. No.: US 2024/0398341 A1
(43) Pub. Date: Dec. 5, 2024

(54) Device for measuring energy expenditures and calculating energy intakes and balances of humans and air-breathing animals

(71) Applicants: Benedict de St. Amatus, Forest Glen (AU); Alexander de St. Amatus, Forest Glen (AU)

(72) Inventors: Benedict de St. Amatus, Forest Glen (AU); Alexander de St. Amatus, Forest Glen (AU)

(21) Appl. No.: 18/205,504

(22) Filed: Jun. 3, 2023

(51) Int. Cl. A61B 5/00; A61B 5/091; A61B 7/00

(52) U.S. Cl. CPC A61B 5/4866; A61B 5/091; A61B 5/4884; A61B 7/003; A61B 5/7475; A61B 2503/40; A61B 2560/0223

(57) ABSTRACT

This invention pertains to a portable device for individualised measurements of energy expenditures and calculating energy intakes and balances of humans and air-breathing animals. Said device utilises algorithms that use a relation between the cumulative energy expenditure and the pulmonary and the cutaneous respirations of a user of said device; and a relation between said pulmonary tidal volume and at least one of the respiration and the hearth frequencies of said user; and a relation between the cumulative energy intake and the masses of carbohydrates, fats and proteins in the dietary intake of said user; wherein the energy balance of said user is the difference between the cumulative energy expenditure and the cumulative energy intake. Data received from a pulse frequency sensor is sufficient for routine operation of said device.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### FIELD OF THE INVENTION

This invention pertains to the fields of medical and veterinary devices, and more particularly, to the field of portable medical devices for measurement of the energy expenditures and calculation of the energy intakes and balances of humans; and to the field of portable veterinary devices for measurement of the energy expenditures and calculation of the energy intakes and balances of air-breathing animals.

### DESCRIPTION OF RELATED ART

The reference in this specification to any prior publication (or information derived from it), or to any matter which is known, is not, and should not be taken as an acknowledgment or admission or any form of suggestion, that the prior publication (or information derived from it) or known matter forms part of the common general knowledge in the field of endeavour, to which this specification relates.

Humans and the air-breathing animals metabolise carbohydrates, fats and proteins to generate energy, using atmospheric oxygen as an oxidiser. The products of the metabolism are carbon dioxide and water. The process of gaseous exchange is known as respiration. When the gaseous exchange occurs through the membranes of the lungs, it is known as pulmonary respiration. When the gaseous exchange occurs through the skin, it is known as cutaneous respiration. The gaseous products of the metabolism are expelled at relatively constant concentrations. The parameters of the pulmonary and the cutaneous respirations may differ for the different air-breathing species, depending on their anatomy and metabolism.

With an exception for the growing organisms of the humans and the air-breathing animals, the energy intake in form of food for a period of time must equalise the energy expenditure for the same period of time. An imbalance may result in starvation and death, or in obesity and health complications, possibly also resulting in death.

Said energy intake can be found by multiplying tabulated data on the calorific value of each type of food consumed for a period of time by the measured mass of said food.

The energy expenditure in humans and air-breathing animals can be calculated either from the amount of oxygen consumed, or from the amount of carbon dioxide produced per unit time, providing that the energy resulting from the oxidation process of the three basic types of foods (carbohydrates, fats and proteins) per unit volume of the oxygen or the carbon dioxide is known. For example, the equation E=|ΔC|W(a_{CH}X_{CH}+a_{fat}X_{fat}+aₚᵣₒₜXₚᵣₒₜ) proposed by Karlberg (1952) allows for calculation of the energy expenditure. Here, E is energy expenditure, |ΔC| is a constant representing the absolute value of the concentration change of carbon dioxide or oxygen; W is the volume of the inhaled or exhaled air, also known as the pulmonary tidal volume, or simply—the tidal volume; a_{CH}, a_{fat} and aₚᵣₒₜ are the calorific values of the carbohydrates, fats and proteins per litter of oxygen consumed; X_{CH}, X_{fat} and Xₚᵣₒₜ are the ratios of carbohydrates, fats and proteins in the dietary intake.

AU patent 2017101440 discloses a method and apparatus for calorimetry in humans and air-breathing animals, in which it is assumed that |ΔC|, a_{CH}, a_{fat} and aₚᵣₒₜ are constant and the energy expenditure of the user can be calculated by only inputting data on measured pulmonary tidal volume and the ratios of carbohydrates, fats and proteins in the dietary intake of said user.

A common method of measuring the tidal volume to apply a mask covering the mouth and nose of the tested user and to measure the velocity of the inhaled or exhaled air for a period of time. This can be done, for example, by a using a spirometer.

Systems comprising spirometers and gas-analysers are used in clinics for measurements of the energy expenditure of the tested user during different activities (run, works, sleep, etc.). However, despite being sufficiently accurate, these systems have significant disadvantages: (i) they require wearing oral-nasal masks by the user; (ii) being expensive and technically complicated, they require permanent connection to the spirometer and the gas analyser during the usage; and (iii) the measurements are performed in clinical conditions which limits the opportunity for everyday monitoring of the energy expenditure. Hence, the existing systems for accurate monitoring the energy consumption by the measuring the volume of the inhaled or exhaled air are inconvenient for a prolonged usage and they are suitable predominantly for clinical purposes.

The invention disclosed in AU patent 2017101440 provides a solution to the problems of technical complexity and inconvenience for everyday usage of the existing devises for measurement of the energy consumption of human bodies. More specifically, the invention disclosed in AU patent 2017101440 eliminates the need for a permanent connection to a spirometer and a gas-analyser, as well as wearing oral-nasal masks during the routine, everyday use of the weight controller.

The operation of the calorimetry apparatus disclosed in AU patent 2017101440 is based on the assumption that for each individual, there is a unique correlation between the measured volumes of the inhaled or exhaled air during a respiration cycle, and the characteristic changes to the time, intensity and frequency spectrum of the recorded breath sounds of said individual.

The calorimetry apparatus disclosed in AU patent 2017101440 comprises a spirometer that measures the tidal volume, a sensitive microphone that is used to record the breath sounds and a programmable processor that is coupled to a controlling interface, a display, and a storage of digital data. Alternatively, a smart phone connected to the spirometer and the microphone can be used to store, process and display the data. The programmable processor utilises two sub-algorithms: (i) a calibration sub-algorithm, which is used to establish a correlation between the measured tidal volume and the recorded breath sounds, in a form of an equation that fits a calibration curve; and (ii) a reverse sub-algorithm, which uses the equation fitting the calibration curve to find the levels of energy consumption that correspond to particular breath sound patterns. The spirometer, in combination with the microphone is used only during the calibration stage. The routine, post-calibration stage requires using only a microphone recording the breath sounds.

Pluralities of portable energy expenditure measuring devices operate using algorithms for calculating the energy expenditure estimates of a user, in which said algorithms estimate the basal metabolic rate of said user and add an estimate of his or her activity data. Said energy expenditure estimates are based on values that a typical for a cohort of people with height, weight, sex, and age similar to those of said user. Therefore, these energy expenditure estimates are correct for said cohort of users, rather than accurately matching the energy expenditure of an individual member of said cohort. AU patent 2017101440 discloses a calorimetry apparatus that is calibrated to the body parameters of the individual user and therefore provides individualised measurements of the energy expenditure of said user.

However, the performance of the calorimetry apparatus disclosed in AU patent 2017101440 is affected by the presence of surrounding noise, which at certain critical intensity levels may cause unreadability or absence of the signals that are coming from the sensitive microphone that is used to record the breath sounds. Also, the intensity of the signals coming from said sensitive microphone weakens with the increase of the distance between said microphone and the source of the breathing sounds, which at certain critical distance may cause unreadability or absence of said signals. Furthermore, wearing said microphone may be inconvenient to some user of said calorimetry apparatus.

Also, AU patent 2017101440 does not disclose an algorithm for calculating the energy intakes of humans and air-breathing animals.

Also, AU patent 2017101440 does not disclose an algorithm for calculating the energy expenditures of humans and air-breathing animals, in which the value of the cutaneous respiration is added.

Also, AU patent 2017101440 does not disclose an algorithm for calculating the energy balances of humans and air-breathing animals.

### OBJECT OF THE INVENTION

It is an object of the present invention to overcome, or at least substantially ameliorate, the disadvantages and shortcomings of the prior art of medical and veterinary devices for measurement of the energy expenditure in humans and air-breathing animals.

In particular, the object of the present invention is to provide solutions to the issues of technical complexity and inconvenience for everyday usage of the existing devices for measurement of the energy expenditure in humans and air-breathing animals. More specifically, the present invention targets elimination of the needs for a permanent connection to a spirometer and a gas-analyser, and for wearing oral-nasal masks during the routine, everyday usage of said devices.

Another object of the present invention is to provide a solution to the issue of lack of accuracy of the existing devices for measurement of the energy expenditures, which inaccuracy is caused by using algorithms that are based on estimates of the averaged energy expenditures of cohorts of users, and for this reason are unable to make individualised, accurate measurements of said energy expenditures.

Yet, another object of the present invention is to provide a solution to the issue of unreadability of auscultation data input or absence of said data input to the algorithms for calculation of the energy balances, which unreadability or absence of data input is caused by presence of surrounding noise or by unsuitably located breath sounds capturing sensors.

Other objects of the present invention are to provide a solution to the issue of absence in the existing algorithms used for measuring the energy expenditures and calculating the energy intakes and the energy balances of the humans and air-breathing animals of terms for calculating the energy intakes, and for adding the values of the cutaneous respiration, and for calculating the energy balances of said humans and air-breathing animals.

Other objects and advantages of the present invention will become apparent from the following description, taken in connection with the accompanying drawings, wherein, by way of illustration and example, embodiments of the present invention are disclosed.

### SUMMARY OF THE INVENTION

According to the present invention, the device for measuring energy expenditures and calculating energy intakes and balances of humans and air-breathing animals comprises a digital data processing unit, a digital memory storage unit, a power supply unit, a user interface unit, a cumulative tidal volume measuring unit, and at least one of a respiration frequency unit and a hearth frequency unit.

The digital data processing unit of said device runs algorithms written in a computer programming language that use a relation between the cumulative energy expenditure and the pulmonary and the cutaneous respirations of a user of said device and a relation between the cumulative energy intake and the masses of carbohydrates, fats and proteins in the dietary intake of said user.

In the relation between the cumulative energy expenditure and the pulmonary and the cutaneous respirations of a user of said device, the cumulative energy expenditure for a period of time equals to a sum of the calorific values of the carbohydrates, fats and proteins per litter of oxygen that are each multiplied by the respective ratios of carbohydrates, fats and proteins in the dietary intake of said user for said period of time; and said sum is multiplied by the concentration of oxygen in the atmosphere, and the absolute value of the concentration change of carbon dioxide or oxygen in the air inhaled or exhaled by said user, and the pulmonary tidal volume of said user for said period of time to which a value for the respective cutaneous respiration of said user is added.

In the relation between the cumulative energy expenditure and the pulmonary and the cutaneous respirations of a user of said device there are constant terms including the concentration of oxygen in the atmosphere, and the absolute value of the concentration change of carbon dioxide or oxygen in the air inhaled or exhaled by said user, and the calorific values of the carbohydrates, fats and proteins per litter of oxygen, and the ratio of the pulmonary to the cutaneous respiration of said user; and there are variable terms including the masses of carbohydrates, fats and proteins in the dietary intake of said user and the pulmonary tidal volume of said user for said period of time.

In the relation between the cumulative energy expenditure and the pulmonary and the cutaneous respirations of a user of said device, there is a relation between the pulmonary tidal volume of said user and at least one of the respiration and the hearth frequencies of said user.

In the relation between the cumulative energy intake and the masses of carbohydrates, fats and proteins in the dietary intake of a user of said device, said cumulative energy intake for a period of time equals to a sum of terms, in which constant calorific values per unit of mass of the carbohydrates, fats and proteins are each multiplied by the respective variable masses of carbohydrates, fats and proteins in the dietary intake of said user for said period of time.

The digital data processing unit of said device is configured to run a calibration algorithm written in a computer programming language, in which a correlation is obtained in a form of a calibration equation that fits data points of the cumulative tidal volume, and at least one of the respiration frequency and the heartbeat frequency that are measured at at least two levels of physical stress of a user of said device.

The digital data processing unit of said device is configured to run a reverse algorithm written in a computer programming language, in which the calibration equation uses at least one of the respiration frequency and the heartbeat frequency that are measured during a routine usage by a user of said device to calculate the cumulative tidal volume of said user for a period of time; and in which said cumulative tidal volume and the masses of carbohydrates, fats and proteins in the dietary intake of said user are used to calculate at least one of the cumulative energy expenditure and the cumulative energy intake and the energy balance of said user for said period of time; and in which warning signals are generated in cases of energy imbalance of said user.

During running the stages of the calibration algorithm, the digital data processing unit is coupled to the digital memory storage unit, the power supply unit, the user interface unit, the cumulative tidal volume measuring unit, and at least one of the respiration frequency unit and the hearth frequency unit. During running the stages of the reverse algorithm, the digital data processing unit is coupled to the digital memory storage unit, and the power supply unit, and the user interface unit, and the cumulative tidal volume measuring unit, and at least one of the respiration frequency unit and the hearth frequency unit.

The digital data processing unit is any unit that is able to transmit data to and from the digital memory storage unit; and to transmit data and commands to and from the user interface unit, and to the cumulative tidal volume measuring unit, and to at least one of the respiration frequency unit and the hearth frequency unit; and that is able to process data from the cumulative tidal volume measuring unit and at least one of the respiration frequency unit and the hearth frequency unit; and that is able to run the written in a computer programming language calibration and reverse algorithms and to produce output data on at least one of the energy intake, and the energy expenditure, and the energy balance of the user of said device; and that is able to transmit the output data to the user interface unit.

The digital memory storage unit is any unit that stores digital data. The user interface unit is any unit that enables entering data and commands to the digital data processing unit; and that is able to transmit data and commands to and from said digital data processing unit; and that is able to display at least one of the energy expenditure and the energy intake and the energy balance of a user of said device; and that is able to send warning signals in cases of energy imbalance of said user. The cumulative tidal volume measuring unit is any unit that is able to measure the total volume per unit time of the inhaled or the exhaled air from the lungs of a user of said device. The respiration frequency unit is any unit that is able to auscultate lungs' sounds of a user of said device during the inhalation and the exhalation periods of a respiration cycle of said user, and that is able to transmit the auscultation data to the digital data processing unit. The hearth frequency unit is any unit that is able to capture heartbeats signals of a user of said device, and that is able to transmit the heartbeats signals to the digital data processing unit.

The auscultated by the respiration frequency unit signals from the lungs' sounds of the user of said device have an intensity level spike at the beginning of the inhalation period and an intensity level spike at the beginning of the exhalation period of the respiration cycle of said user, which intensity level spikes are distinguishable by their duration, level of intensity and location within the timeline of the respiration cycle, and the number of said intensity level spikes per unit time is counted by the digital data processing unit to find the respiration frequency of said user. The signals captured by the hearth frequency unit have varying intensity levels with a single intensity level spike per a heartbeat of a user of said device, and the digital data processing unit counts the number of intensity level spikes per unit time to find the hearth frequency of said user.

Said device operates with input data from the cumulative tidal volume unit and the hearth frequency unit when the respiration frequency unit is absent. Hearth frequency data is used to at least one of the functions of supplementing and validation the respiration rate data when the data from the respiration frequency unit is unreadable.

Different sets of dimensions of said device are used to fit the anatomical features of each of the air-breathing species; and different sets of constants are used to calculate the cumulative energy expenditure and the cumulative energy intake of said air-breathing species, depending on metabolisms specifics of said air-breathing species.

### BRIEF DESCRIPTION OF THE DRAWING

By way of illustration only, an embodiment of the invention is described more fully hereinafter with reference to the accompanying drawings, in which:

FIG. 1 shows an embodiment of a device for measuring energy expenditures and calculating energy intakes and balances of humans and air-breathing animals used during running the stages of the calibration algorithm, in which the data inputs to the calibration algorithm of said device are from a cumulative tidal volume measuring unit, a respiration frequency unit and a hearth frequency unit.

FIG. 2 shows an embodiment of a device for measuring energy expenditures and calculating energy intakes and balances of humans and air-breathing animals used during running the stages of the calibration algorithm, in which auscultation data from the respiration frequency unit is absent or unreadable and the data inputs to the calibration algorithm of said device are from the hearth frequency unit and the cumulative tidal volume measuring unit only.

FIG. 3 shows an example of the stages of the calibration algorithm.

FIG. 4 shows an example of simultaneous measurements and recording the intensity levels of the bronchial respiration sounds and the hearth signals of a human user of the device for measuring the energy expenditures and calculating the energy intakes and balances.

FIG. 5 shows the location of the gradients ΔIᵢ/ΔPᵢ and ΔIₑ/ΔPₑ on the intensity level I vs. time t chart.

FIG. 6 shows an example chart of the data points of the respiration and the hearth frequencies vs. the cumulative tidal volumes of a human user of the device for measuring the energy expenditures and calculating the energy intakes and balances.

FIG. 7 shows an example of curves fitting data points of the respiration and hearth frequencies.

FIG. 8 shows an embodiment of the device for measuring energy expenditures and calculating energy intakes and balances of humans and air-breathing animals for routine usage of said device, in which the data inputs to the reverse algorithm of said device are from the respiration frequency and the hearth frequency units.

FIG. 9 shows an embodiment of the device for measuring energy expenditures and calculating energy intakes and balances of humans and air-breathing animals for routine usage of said device, in which auscultation data from the respiration frequency unit is absent or unreadable and the data input to the reverse algorithm of said device is from the hearth frequency unit only.

FIG. 10 is an example of the stages of the reverse algorithm.

### DETAILED DESCRIPTION OF THE INVENTION

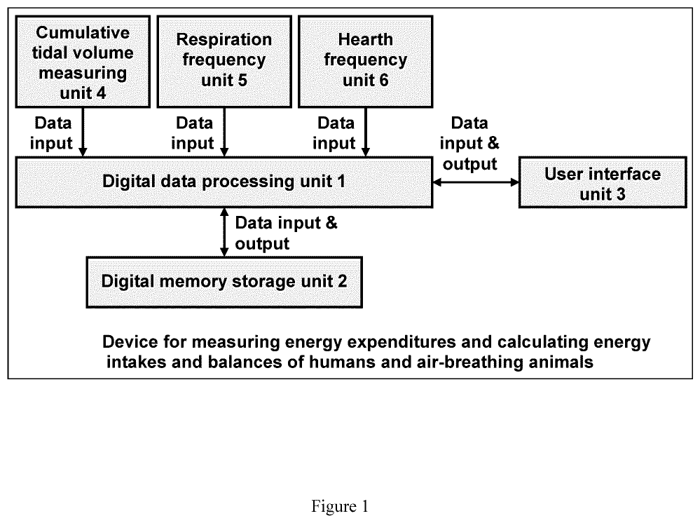

Two distinct embodiments of a device for measuring energy expenditures and calculating energy intakes and balances of humans and air-breathing animals according to the present invention are shown with references to FIGS. 1, 2 and 8, 9.

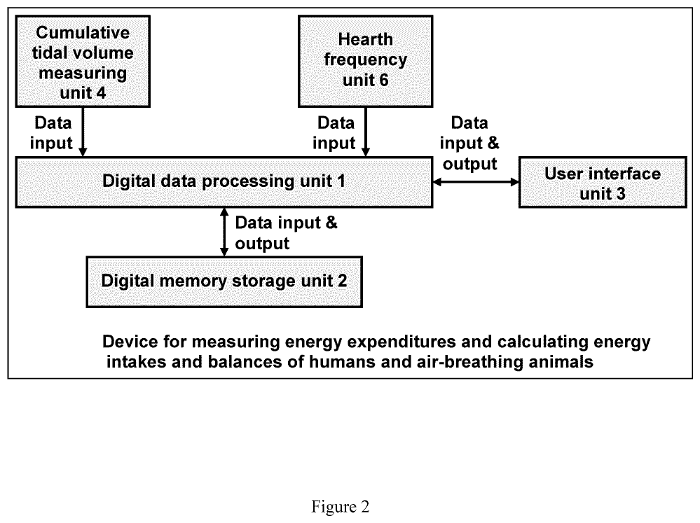

The embodiments shown in FIGS. 1 and 2 are configured to run a calibration algorithm written in a computer programming language. Said calibration algorithm utilises a calibration equation that fits data points of the cumulative tidal volume and at least one of the respiration frequency and the heartbeat frequency that are measured at at least two levels of physical stress of a user of said device.

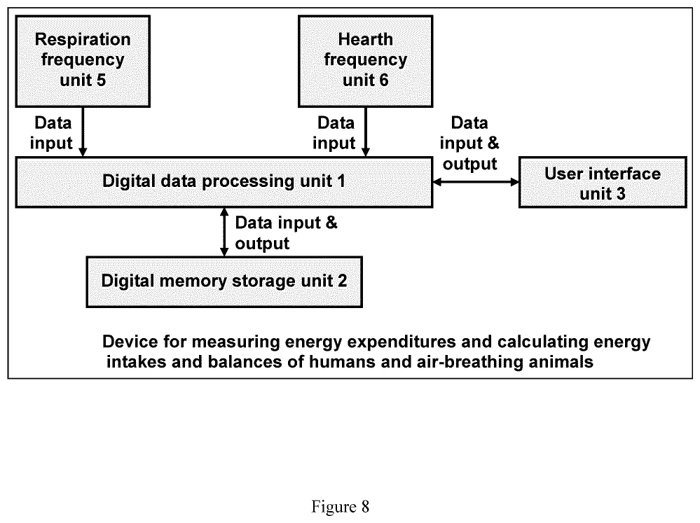

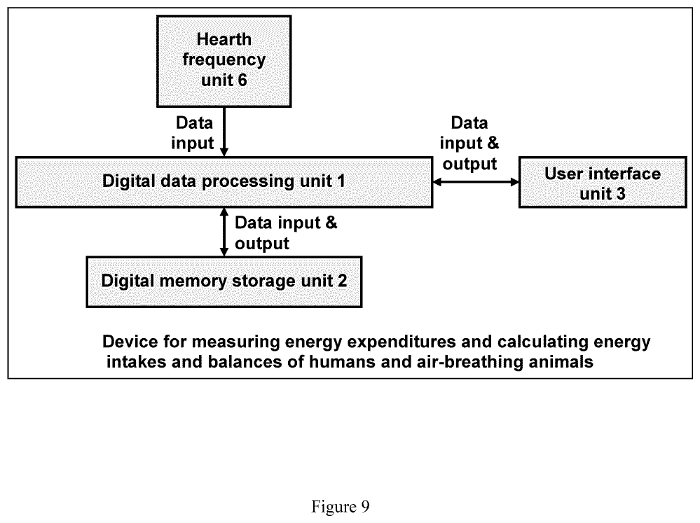

The embodiments shown in FIGS. 8 and 9 are configured to run a reverse algorithm written in a computer programming language, in which said calibration equation is used to find the energy expenditure and balance that correspond to at least one of the respiration frequency and the heartbeat frequency of the user that are measured during the routine use of said device.

The embodiments shown in FIGS. 1, 2 and 8, 9 are applicable to human users and to air-breathing animals, with adjustments to the anatomy of each of the air-breathing species.

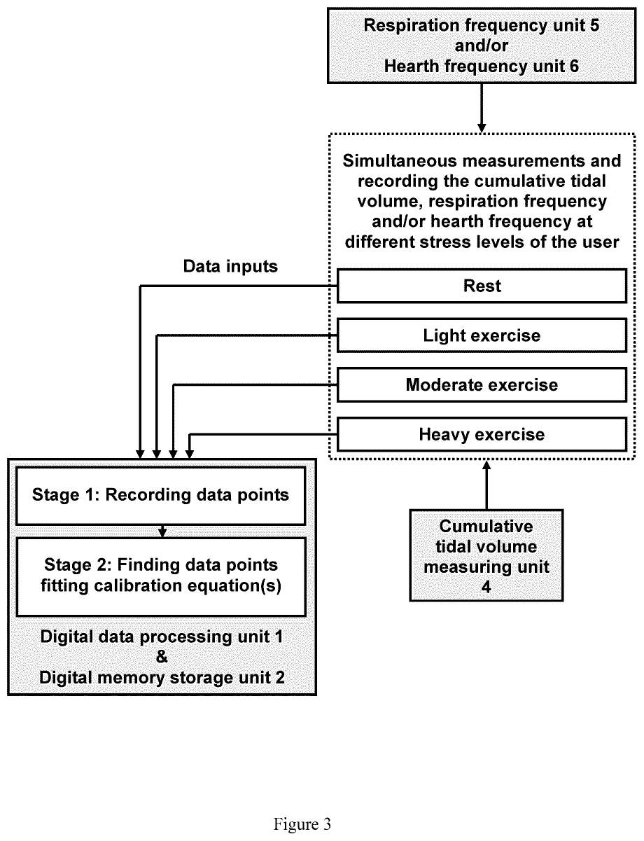

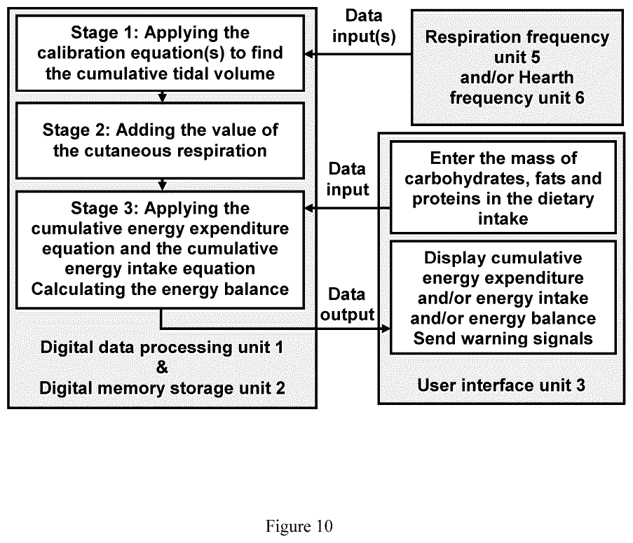

The stages of the calibration algorithm are shown in FIGS. 3 to 7. The stages of the reverse algorithm are shown in FIG. 10. Said calibration and reverse algorithms are applicable to human users and to air-breathing animals.

Accordingly, it is to be understood that the embodiments of the invention and the operational algorithms herein described are merely illustrative of the application of the principles of the invention. References herein to details of the illustrated embodiments are not intended to limit the scope of the claims, which themselves recite those features regarded as essential to the invention.

In the embodiment shown in FIG. 1, the device for measuring energy expenditures and calculating energy intakes and balances of humans and air-breathing animals 1 (hereinafter Device) used during running the stages of the calibration algorithm, comprises a digital data processing unit 1 (hereinafter Unit 1) that is coupled to a digital memory storage unit 2 (hereinafter Unit 2), a power supply unit (not shown in the figures), and a user interface unit 3 (hereinafter Unit 3). Said Unit 1 receives and processes data from a cumulative tidal volume measuring unit 4 (hereinafter Unit 4), auscultation data from a respiration frequency unit 5 (hereinafter Unit 5), wherein the respiration frequency is the number of respiration cycles per unit time, and data from a hearth frequency unit 6 (hereinafter Unit 6), wherein the hearth frequency is the number of heartbeats per unit time. Said Units 4, 5 and 6 are suitably located at positions at or near the body of the user of said Device, wherein the suitability of said locations depends on whether said Units 4, 5 and 6 are able to capture readable and valid data. The data is stored in Unit 2. Unit 3 is used to enter data and commands, and to transmit data and commands to Unit 1, and to display data.

In the embodiment shown in FIG. 1, the data from said Unit 6 is used to at least one of the functions of supplementing and validating the auscultation data from said Unit 5.

Units 4, 5 and 6 are suitably designed to ensure appropriate fitting to the body of each of the air-breathing species.

FIG. 2 shows an embodiment of a Device used during running the stages of the calibration algorithm, in which auscultation data from Unit 5 is absent or unreadable and the calibration algorithm of the Device operates with data received from Unit 6 and Unit 4 only.

In the embodiment shown in FIG. 2, Unit 1 is coupled to Unit 2, the power supply unit, and Unit 3, and receives and processes data from Unit 4 and Unit 6. Said Unit 4 and Unit 6 are suitably located at positions at or near the body of the user of the Device, wherein the suitability of said locations depends on whether said Unit 4 and Unit 6 are able to capture readable and valid data. The data is stored in Unit 2. Unit 3 is used to enter data and commands, and to transmit data and commands to Unit 1, and to display data.

In the embodiments of the Device, said Unit 1 that is coupled to said Unit 2 and the power supply unit, is any unit that is able to transmit data and commands to and from said Unit 3 and said Unit 4 and at least one of said Unit 5 and said Unit 6; and that is able to process data from said Unit 4 and at least one of said Unit 5 and said Unit 6; and that is able to run the written in a computer programming language calibration and reverse algorithms and to produce output data on at least one of the energy intake and the energy expenditure and the energy balance of the user of the Device; and that is able to transmit said output data to Unit 3.

In the embodiments of the Device, said Unit 4 is any unit, as for example a spirometer or an inflatable bag, which is able to measure the volume of the inhaled or the exhaled air from the lungs of the user of said Device per unit time.

In the embodiments of the Device, said Unit 5 is any unit, as for example an assembly of an acoustic amplifier (stethoscope) and a microphone, which is able to auscultate the sounds made by the lungs of the user of said Device, and which is able to transmit the auscultation data to Unit 1.

In the embodiments of the Device, said Unit 6 is any unit, as for example a photoplethysmography sensor, which is able to capture heartbeats signals of the user of said Device, and which is able to transmit the heartbeats signals to Unit 1.

FIG. 3 shows an example of the stages of the calibration algorithm. In this example, said calibration algorithm is executed in two consequent stages.

During Stage 1 of the calibration algorithm, Unit 1 receives simultaneous data from Unit 4, and at least one of Unit 5 and Unit 6. The data from said Unit 4, at least one of said Unit 5 and said Unit 6, is collected at at least two different levels such as rest, light, moderate and heavy level of physical stress of the user of the Device.

The data from Unit 4 is in a format of units of volume per a unit of time. The data from Unit 4 is entered to Unit 1 by means of at least one of the electronic transmission of data between said Unit 4 and said Unit 1 and the manual transmission of data between said Unit 4 and said Unit 1 by using Unit 3. The cumulative tidal volume data is stored in Unit 2.

The auscultation data from Unit 5 is in a format of a record of respiration sounds with varying intensity levels recorded during the respiration cycles of the user of the Device. Unit 1 counts the number of said intensity level spikes per unit time to find the respiration frequency of said user of the Device. The respiration frequency data is stored in Unit 2.

The data from Unit 6 is in a format of a record of signals of varying intensity levels with a single intensity level spike per a heartbeat. Unit 1 counts the number of said intensity level spikes per unit time to find the hearth frequency of the user. The hearth frequency data is stored in Unit 2.

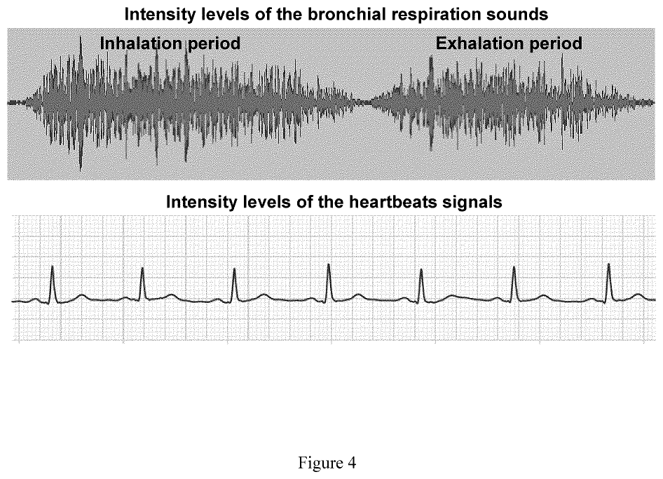

FIG. 4 shows an example of data in format of signals with varying intensity levels that Unit 6 sends to Unit 1. FIG. 4 also shows an example of the varying intensity levels of the bronchial respiration sounds of a human user of the Device during one respiration cycle. There are two intensity level spikes per a single respiration cycle. There is an intensity level spike at the beginning of the inhalation period of the respiration cycle and there is an intensity level spike at the beginning of the exhalation period of the same respiration cycle. The calibration algorithm selects and uses for finding the respiration frequency only one intensity level spike per respiration cycle and that is either the intensity level spike at the beginning of the inhalation period of the respiration cycle or the intensity level spike at the beginning of the exhalation period of the same respiration cycle.

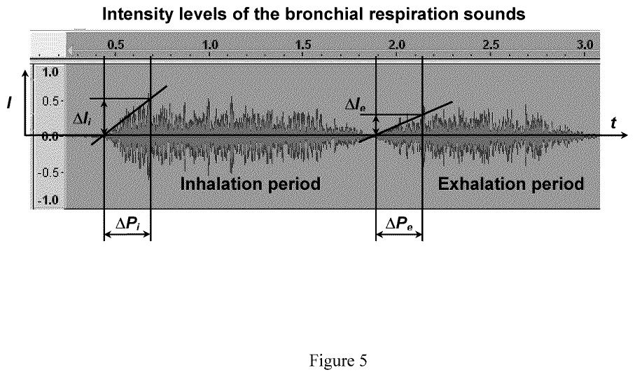

FIG. 5 shows an example, in which the calibration algorithm uses a difference between the gradients ΔIᵢ/ΔPᵢ and ΔIₑ/ΔPₑ to recognise and distinguish the spikes at the beginning of the inhalation period from the spikes at the beginning of the exhalation period. Here ΔIᵢ is the difference between the zero level (or the level of the background noise) of the intensity level at the end of pause between two complete respiration cycles and the time of recording the sound with the maximal intensity level. Similarly, ΔIₑ is the difference between the zero level (or the level of the background noise) of the intensity level at the end of pause between inspiration and expiration periods of the respiration cycle and the maximal intensity level. The pause between two complete respiration cycles is longer than the pause between inspiration and expiration periods of the respiration cycle, which allows the calibration algorithm to distinguish these two types of pauses. ΔPᵢ and ΔPₑ are the two time periods corresponding to ΔIᵢ and ΔIₑ.

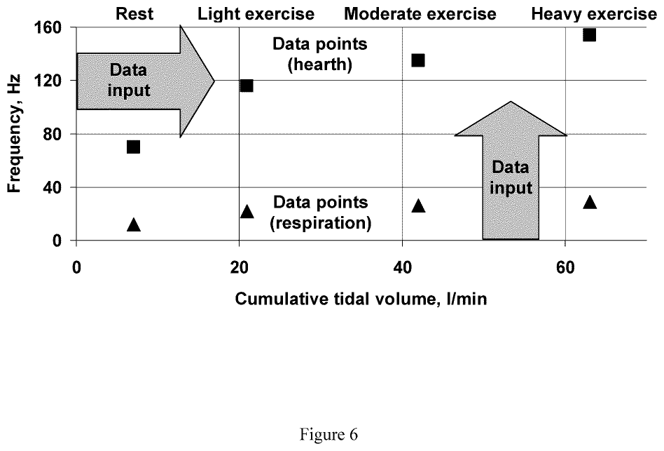

The calibration algorithm tabulates the data collected from Unit 4 and from at least one of Unit 5 and Unit 6, at the different levels of physical stress of the user of the Device. FIG. 6 shows a chart of the data points of the respiration and the hearth frequencies plotted against the cumulative tidal volumes, wherein said data points and said cumulative tidal volumes are simultaneously recorded during the rest and during the light, moderate and heavy exercises carried out by a human user of the Device.

During Stage 2 of the calibration algorithm, said calibration algorithm finds calibration equation(s) of at least one of the types

$${\sum }_{0}^{t} W = f ( {F}_{respiration} ) and {\sum }_{0}^{t} W = f ( {F}_{hearth} )$$

that fit the recorded and tabulated in Stage 1 of said calibration algorithm data points. Here,

$${\sum }_{0}^{t} W$$

is the cumulative tidal volume for the time period from 0 to t, Fᵣₑₛₚᵢᵣₐₜᵢₒₙ and Fₕₑₐᵣₜₕ are, respectively, the respiration and hearth frequencies as recorded at different levels of physical stress of the user of the Device.

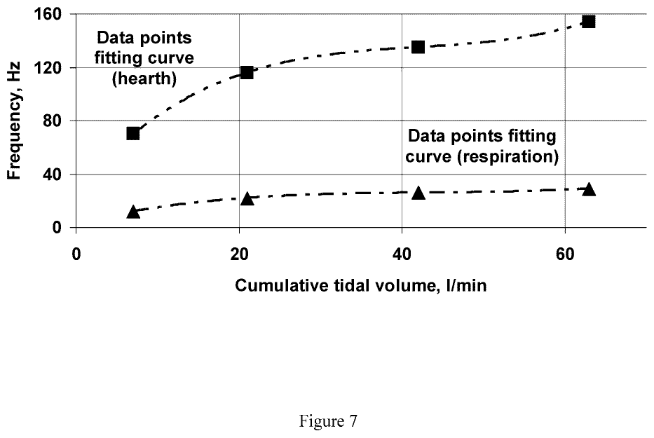

FIG. 7 shows an example of polynomial curves fitting data points of the respiration and hearth frequencies as recorded at different levels of physical stress of the user of the Device.

FIG. 8 shows an embodiment of the Device for routine usage of said Device, in which the reverse algorithm of the Device operates with data received from Units 5 and 6.

In this embodiment, Unit 1 is coupled to Unit 2, the power supply unit, and Unit 3. Said Unit 1 receives and processes auscultation data from Unit 5 and data from Unit 6, which data is used to run the reverse algorithm of the Device. Units 5 and 6 are suitably located at positions at or near the body of the user of the Device, wherein the suitability of said locations depends on whether said Units 5 and 6 are able to capture readable and valid data. The data is stored in Unit 2. Unit 3 is used to enter data and commands, and to transmit data and commands to Unit 1, and to display data.

In this embodiment, the data from said Unit 6 is used to at least one of the functions of supplementing and validating the auscultation data from said Unit 5.

FIG. 9 shows an embodiment of the Device for routine usage of said Device, in which auscultation data from Unit 5 is absent or unreadable and the reverse algorithm of the Device operates with data received from Unit 6 only.

In this embodiment, Unit 1 receives and processes data from Unit 6 only. Unit 6 is suitably located at a position at or near the body of the user of the Device, wherein the suitability of said location depends on whether said Unit 6 is able to capture readable and valid data. The data is stored in Unit 2. Unit 3 is used to enter data and commands, and to transmit data and commands to Unit 1, and to display data.

FIG. 10 shows an example of the stages of the reverse algorithm. In this example, said reverse algorithm is executed in three consequent stages.

During Stage 1 of the reverse algorithm, Unit 1 receives and processes data from at least one of Unit 5 and Unit 6. The data from at least one of said Unit 5 and said Unit 6 is collected during the routine activities of the user of the Device. The reverse algorithm enters the value(s) of at least one of the frequencies Fᵣₑₛₚᵢᵣₐₜᵢₒₙ and Fₕₑₐᵣₜₕ, as recorded during the routine activities of the user of the Device, into at least one of the calibration equation(s)

$${\sum }_{0}^{t} W = f ( {F}_{respiration} ) and {\sum }_{0}^{t} W = f ( {F}_{hearth} ) ,$$

and calculates the corresponding to at least one of the frequencies Fᵣₑₛₚᵢᵣₐₜᵢₒₙ and Fₕₑₐᵣₜₕ value(s) of the cumulative tidal volume

$${\sum }_{0}^{t} W .$$

In cases of significant differences between the values of the cumulative tidal volume found by using the frequencies Fᵣₑₛₚᵢᵣₐₜᵢₒₙ and Fₕₑₐᵣₜₕ, the reverse algorithm uses ether Fᵣₑₛₚᵢᵣₐₜᵢₒₙ or Fₕₑₐᵣₜₕ, or selects a value between said two values.

During Stage 2 of the reverse algorithm, the constant fraction of the cutaneous respiration R_{cut} is added to the value of the calculated cumulative tidal volume

$${\sum }_{0}^{t} W ,$$

totalling the cumulative respiration of the user to

$${\sum }_{0}^{t} W ( 1 + {R}_{cut} ) .$$

During Stage 3 of the reverse algorithm, Unit 3 is used to enter the masses of the carbohydrates, m_{CH}, the fats, m_{fat}, and the proteins mₚᵣₒₜ in the dietary intake of the user of the Device.

During Stage 3 of the reverse algorithm, the cumulative energy expenditure

$${\sum }_{0}^{t} {E}_{out}$$

for a period from 0 to t is calculated using a modified form of the of equation of Karlberg (1952), namely:

$${\sum }_{0}^{t} {E}_{out} == \vert Δ C \vert {C}_{O} ( {a}_{C H} \frac{{m}_{C H}}{{m}_{C H} + {m}_{fat} + {m}_{prot}} + {a}_{fat} \frac{{m}_{fat}}{{m}_{C H} + {m}_{fat} + {m}_{prot}} + {a}_{prot} \frac{{m}_{prot}}{{m}_{C H} + {m}_{fat} + {m}_{prot}} ) {\sum }_{0}^{t} W ( 1 + {R}_{cut} ) ,$$

where C_{O} is the constant concentration of oxygen in the atmosphere and

$$\frac{{m}_{C H}}{{m}_{CH} + {m}_{fat} + {m}_{p r o t}} , \frac{{m}_{fat}}{{m}_{C H} + {m}_{fat} + {m}_{prot}} and \frac{{m}_{p r o t}}{{m}_{C H} + {m}_{fat} + {m}_{prot}}$$

are respectively the ratios of the carbohydrates, the fats and the proteins in the dietary intake of the user of the Device. The values of |ΔC| and R_{cut} constants and the calorific values of the carbohydrates, fats and proteins, a_{CH}, a_{fat} and aₚᵣₒₜ may differ depending on the anatomy of the different air-breathing species and their metabolism.

For example, in calculating

$${\sum }_{0}^{t} {E}_{out}$$

for humans, |ΔC| may have the value of 3.5% (vol.) as per the data shown in Marrieb (2000); C_{O} is typically 20.95% (vol.) throughout the Earth's atmosphere; R_{cut} may have the value of 1.4% as per the data shown in Fitzgerald (1957) and Alkalay et al. (1971); and the calorific values of the carbohydrates, fats and proteins per litter of oxygen consumed are a_{CH}=5.047 Cal/l, a_{fat}=4.686 Cal/l and aₚᵣₒₜ=4.485 Cal/l respectively as per the data shown in Karlberg (1952).

Also, during Stage 3, the reverse algorithm calculates the cumulative energy intake of the user

$${\sum }_{0}^{t} {E}_{i n}$$

for the period from 0 to t, namely

$${\sum }_{0}^{t} {E}_{i n} = {b}_{C H} {m}_{C H} + {b}_{fat} {m}_{fat} + {b}_{prot} {m}_{prot} ,$$

where b_{CH}, b_{fat} and bₚᵣₒₜ are respectively the calorific values of the carbohydrates, fats and proteins per unit of mass.

Also, during Stage 3, the reverse algorithm compares the values of

$${\sum }_{0}^{t} {E}_{out} and {\sum }_{0}^{t} {E}_{i n} ,$$

wherein any difference of said values, except in cases of growing organisms of the users of the Device, indicates energy imbalance.

In use, during the calibration of the Device, the user completes a set of exercises, using said Device to simultaneously record and process data from Unit 4 and from at least one of Unit 5 and Unit 6. Then, during the routine use of the Device, the user is disconnected from Unit 4, however continues using said Device to record and process data from at least one of said Unit 5 and said Unit 6. Unit 3 is used to enter the masses of the carbohydrates, fats and proteins consumed by the user of said Device for the period of time of routine use of said Device. Unit 3 is also used to display at least one of the cumulative energy expenditure and the cumulative energy intake and the energy balance, and to send warning signals in cases of energy imbalance.

### CITATION LIST

Alkalay, L., Suetsugu, S., Constantine, H., Stein, M. (1971). Carbon dioxide elimination across human skin. American Journal of Physiology 220, pp. 1434-1436.
AU patent 2017101440 “Method and apparatus for calorimetry in humans and air-breathing animals”.
Fitzgerald, L. R. (1957). Cutaneous respiration in man. Physiological Reviews 37, pp. 325-336.
Karlberg P. (1952). Method of calculating the energy metabolism. Acta Paediatrica 41, pp. 67-76.
Marrieb, E. N. (2000). Human Anatomy and Physiology, (5^{th} edition), New York, USA, Benjamin/Cummings, 1237.

The claims defining the invention are as follows:

1. A device for measuring energy expenditures and calculating energy intakes and balances of humans and air-breathing animals comprising:
a digital data processing unit,
a digital memory storage unit,
a power supply unit,
a user interface unit,
a cumulative tidal volume measuring unit,
at least one of a respiration frequency unit and a hearth frequency unit,
wherein the digital data processing unit of said device runs algorithms written in a computer programming language that use a relation between the cumulative energy expenditure and the pulmonary and cutaneous respiration of a user of said device and a relation between the cumulative energy intake and the masses of carbohydrates, fats and proteins in the dietary intake of said user.

2. The device for measuring energy expenditures and calculating energy intakes and balances of humans and air-breathing animals according to claim 1, wherein in the relation between the cumulative energy expenditure and the pulmonary and cutaneous respiration of a user of said device, the cumulative energy expenditure for a period of time equals to a sum of the calorific values of the carbohydrates, fats and proteins per litter of oxygen that are each multiplied by the respective ratios of carbohydrates, fats and proteins in the dietary intake of said user for said period of time; and said sum is multiplied by the concentration of oxygen in the atmosphere, and the absolute value of the concentration change of carbon dioxide or oxygen in the air inhaled or exhaled by said user, and the pulmonary tidal volume of said user for said period of time to which a value for the respective cutaneous respiration of said user is added.

3. The device for measuring energy expenditures and calculating energy intakes and balances of humans and air-breathing animals of claim 1 or claim 2, wherein in the relation between the cumulative energy expenditure and the pulmonary and cutaneous respiration of a user of said device there are constant terms including the concentration of oxygen in the atmosphere, and the absolute value of the concentration change of carbon dioxide or oxygen in the air inhaled or exhaled by said user, and the calorific values of the carbohydrates, fats and proteins per litter of oxygen, and the ratio of the pulmonary to cutaneous respiration of said user; and there are variable terms including the masses of carbohydrates, fats and proteins in the dietary intake of said user and the pulmonary tidal volume of said user for said period of time.

4. The device for measuring energy expenditures and calculating energy intakes and balances of humans and air-breathing animals according to any one of the preceding claims, wherein in the relation between the cumulative energy expenditure and the pulmonary and cutaneous respiration of a user of said device, there is a relation between the pulmonary tidal volume of said user and at least one of the respiration and the hearth frequencies of said user.

5. The device for measuring energy expenditures and calculating energy intakes and balances of humans and air-breathing animals according to any one of the preceding claims, wherein in the relation between the cumulative energy intake and the masses of carbohydrates, fats and proteins in the dietary intake of a user of said device, said cumulative energy intake for a period of time equals to a sum of terms, in which constant calorific values per unit of mass of the carbohydrates, fats and proteins are each multiplied by the respective variable masses of carbohydrates, fats and proteins in the dietary intake of said user for said period of time.

6. The device for measuring energy expenditures and calculating energy intakes and balances of humans and air-breathing animals according to claim 1, wherein the digital data processing unit of said device is configured to run a calibration algorithm written in a computer programming language, in which a correlation is obtained in a form of a calibration equation that fits data points of the cumulative tidal volume, and at least one of the respiration frequency and the heartbeat frequency that are measured at at least two levels of physical stress of a user of said device.

7. The device for measuring energy expenditures and calculating energy intakes and balances of humans and air-breathing animals of claim 1 or claim 2,, wherein the digital data processing unit of said device is configured to run a reverse algorithm written in a computer programming language, in which the calibration equation uses at least one of the respiration frequency and the heartbeat frequency that are measured during a routine usage by a user of said device to calculate the cumulative tidal volume of said user for a period of time; and in which said cumulative tidal volume and the masses of carbohydrates, fats and proteins in the dietary intake of said user are used to calculate at least one of the cumulative energy expenditure and the cumulative energy intake and the energy balance of said user for said period of time; and in which warning signals are generated in cases of energy imbalance of said user.

8. The device for measuring energy expenditures and calculating energy intakes and balances of humans and air-breathing animals according to any one of the preceding claims, wherein during running the stages of the calibration algorithm, the digital data processing unit is coupled to the digital memory storage unit, the power supply unit, the user interface unit, the cumulative tidal volume measuring unit, and at least one of the respiration frequency unit and the hearth frequency unit.

9. The device for measuring energy expenditures and calculating energy intakes and balances of humans and air-breathing animals according to any one of the preceding claims, wherein during running the stages of the reverse algorithm, the digital data processing unit is coupled to the digital memory storage unit, and the power supply unit, and the user interface unit, and the cumulative tidal volume measuring unit, and at least one of the respiration frequency unit and the hearth frequency unit.

10. The device for measuring energy expenditures and calculating energy intakes and balances of humans and air-breathing animals according to any one of the preceding claims, wherein the digital data processing unit is any unit that is able to transmit data to and from the digital memory storage unit; and to transmit data and commands to and from the user interface unit, and to the cumulative tidal volume measuring unit, and to at least one of the respiration frequency unit and the hearth frequency unit; and that is able to process data from the cumulative tidal volume measuring unit and at least one of the respiration frequency unit and the hearth frequency unit; and that is able to run the written in a computer programming language calibration and reverse algorithms and to produce output data on at least one of the energy intake, and the energy expenditure, and the energy balance of the user of said device; and that is able to transmit the output data to the user interface unit.

11. The device for measuring energy expenditures and calculating energy intakes and balances of humans and air-breathing animals according to any one of the preceding claims, wherein the digital memory storage unit is any unit that stores digital data.

12. The device for measuring energy expenditures and calculating energy intakes and balances of humans and air-breathing animals according to any one of the preceding claims, wherein the user interface unit is any unit that enables entering data and commands to the digital data processing unit; and that is able to transmit data and commands to and from said digital data processing unit; and that is able to display at least one of the energy expenditure and the energy intake and the energy balance of a user of said device; and that is able to send warning signals in cases of energy imbalance of said user.

13. The device for measuring energy expenditures and calculating energy intakes and balances of humans and air-breathing animals according to any one of the preceding claims, wherein the cumulative tidal volume measuring unit is any unit that is able to measure the total volume per unit time of the inhaled or the exhaled air from the lungs of a user of said device.

14. The device for measuring energy expenditures and calculating energy intakes and balances of humans and air-breathing animals according to any one of the preceding claims, wherein the respiration frequency unit is any unit that is able to auscultate lungs' sounds of a user of said device during the inhalation and the exhalation periods of a respiration cycle of said user, and that is able to transmit the auscultation data to the digital data processing unit.

15. The device for measuring energy expenditures and calculating energy intakes and balances of humans and air-breathing animals according to any one of the preceding claims, wherein the auscultated by the respiration frequency unit signals from the lungs' sounds of the user of said device have an intensity level spike at the beginning of the inhalation period and an intensity level spike at the beginning of the exhalation period of the respiration cycle of said user, which intensity level spikes are distinguishable by their duration, level of intensity and location within the timeline of the respiration cycle, and the number of said intensity level spikes per unit time is counted by the digital data processing unit to find the respiration frequency of said user.

16. The device for measuring energy expenditures and calculating energy intakes and balances of humans and air-breathing animals according to any one of the preceding claims, wherein the hearth frequency unit is any unit that is able to capture heartbeats signals of a user of said device, and that is able to transmit the heartbeats signals to the digital data processing unit.

17. The device for measuring energy expenditures and calculating energy intakes and balances of humans and air-breathing animals according to any one of the preceding claims, wherein the signals captured by the hearth frequency unit have varying intensity levels with a single intensity level spike per a heartbeat of a user of said device, and the digital data processing unit counts the number of intensity level spikes per unit time to find the hearth frequency of said user.

18. The device for measuring energy expenditures and calculating energy intakes and balances of humans and air-breathing animals according to any one of the preceding claims, wherein said device operates with input data from the cumulative tidal volume unit and the hearth frequency unit when the respiration frequency unit is absent.

19. The device for measuring energy expenditures and calculating energy intakes and balances of humans and air-breathing animals according to any one of the preceding claims, wherein hearth frequency data is used to at least one of the functions of supplementing and validation the respiration rate data when the data from the respiration frequency unit is unreadable.

20. The device for measuring energy expenditures and calculating energy intakes and balances of humans and air-breathing animals according to any one of the preceding claims, wherein different sets of dimensions of said device are used to fit the anatomical features of each of the air-breathing species; and different sets of constants are used to calculate the cumulative energy expenditure and the cumulative energy intake of said air-breathing species, depending on metabolisms specifics of said air-breathing species.
